(19) United States
(12) Patent Application Publication
(10) Pub. No.: US 2025/0386884 A1
(43) Pub. Date: Dec. 25, 2025

(54) MOISTURE-ABSORBING GUARD

(71) Applicant: Jocelyn Marie HUGHES, Silver Spring, MD (US)

(72) Inventor: Jocelyn Marie HUGHES, Silver Spring, MD (US)

(21) Appl. No.: 19/242,532

(22) Filed: Jun. 18, 2025

Related U.S. Application Data
(60) Provisional application No. 63/662,116, filed on Jun. 20, 2024.
(60) Provisional application No. 63/715,590, filed on Nov. 3, 2024.

(51) Int. Cl. A41D 13/08

(52) U.S. Cl. CPC A41D 13/08; A41D 2400/00

(57) ABSTRACT

Disclosed is a moisture-absorbing guard having an outer absorbent layer having a network of interconnected pores, a base adhesive layer, and a moisture-locking interior layer between the outer absorbent layer and the base adhesive layer, the moisture-locking interior layer having an absorbent material. Also disclosed is a method of using a moisture-absorbing guard as described.

### CROSS-REFERENCE TO RELATED APPLICATIONS

This application claims priority to U.S. Provisional Application No. 63/662, 116,filed Jun. 20, 2024, and U.S. Provisional Application No. 63/715,590, filed Nov. 3, 2024, the contents of both applications being incorporated by reference herein in their entireties.

### TECHNICAL FIELD

This disclosure pertains to cosmetic and beauty care, focusing on a guard that protects makeup and topicals applied to the face and body from tears, sweat, and facial oils. It is designed for use in industries such as cosmetics, sports/athletics, and hospitality, where maintaining a polished, professional appearance is crucial.

### Background

This disclosure relates to personal care products specifically designed for the cosmetic and beauty industry, addressing the critical need to preserve makeup and skincare topical integrity in high-moisture environments. The present disclosure is a moisture-absorbing guard designed to absorb lacrimal secretions (teardrops), hidrosis (sweat), and facial oils, ensuring that makeup and skincare topicals applied to the face and body remain intact, flawless, and long-lasting. The guard may be designed to be applied/adhered only to clean, product-free, non-facial, non-sensitive areas such as the fingers, knuckles, wrists, forearms, biceps, and shoulder area, allowing the user to discreetly dab moisture from the face and body. By capturing moisture at the source, the guard prevents it from reaching and disrupting makeup or topicals, stopping the breakdown of cosmetic products that would otherwise lead to smudging, fading, or the need for frequent reapplication.

Despite advancements in long-wear makeup formulas claiming 24-hour durability, makeup wearers continue to experience degradation throughout the day due to natural skin oils, sweat, tears, environmental factors, and physical contact. These elements break down makeup, causing it to fade, shift, or become uneven. Existing moisture-management products fail to meet the specific needs of makeup wearers, as they are primarily designed for hygiene or sports applications and do not prevent cosmetic degradation in high-moisture environments like sporting events, food service, or emotionally charged settings such as weddings or therapy sessions. The guard of the present disclosure absorbs moisture upon contact, ensuring that makeup and topicals remain intact.

### SUMMARY

The present disclosure relates to a multi-layer, moisture-absorbing guard designed to protect the integrity of applied makeup and skincare topicals to the face and body in high-moisture conditions.

### BRIEF DESCRIPTION OF THE DRAWINGS

FIG. 1 shows an example plan/top view of the guard of the present disclosure.

FIG. 2 shows an example sectional view of the guard of the present disclosure.

FIG. 3 shows an example exploded view of the guard of the present disclosure.

FIG. 4 shows example guards of the present disclosure as worn by a user.

FIG. 5 shows example guards of the present disclosure as worn by a user.

FIG. 6 shows example guards of the present disclosure as worn by a user.

FIG. 7 shows example guards of the present disclosure as worn by a user.

FIG. 8 shows example guards of the present disclosure as worn by a user.

### DETAILED DESCRIPTION

The present disclosure relates to a multi-layer, moisture-absorbing guard designed to protect the integrity of applied makeup and skincare topicals to the face and body in high-moisture conditions. The guard comprises a unique three-layer structure, offering specific advantages in moisture management, adhesion, and discreet usage. Each layer of the guard has been engineered to perform a distinct function, working together to absorb, retain, and prevent moisture from disrupting applied makeup and skincare topicals. The disclosure's design provides long-lasting protection for users in various high-moisture environments, such as weddings, performances, and extended work shifts, enabling them to maintain a polished appearance without frequent makeup touch-ups or reapplication.

The guard of the present disclosure may include two, three, four, or more layers. According to some aspects, the guard may include an outer absorbent layer, a moisture-locking interior layer, a base adhesive layer, a removable protective liner, or a combination thereof.

The outer absorbent layer of the guard may include moisture-wicking properties, designed to absorb fluids such as sweat, tears, and facial oils upon contact. Constructed from hydrophilic, oleophilic, and/or amphiphilic materials-such as cotton, cellulose, nylon, PVA, other cosmetic-grade fibers, and combinations thereof-this layer captures moisture before it interacts with applied makeup or topicals. The outer absorbent layer may be treated to enhance its hydrophilicity and oleophilicity, and may actively wick moisture away from the skin, preventing makeup degradation. Microscopic pores may be distributed uniformly across the surface, allowing for breathability while supporting continuous moisture absorption, maintaining the guard's effectiveness without saturation. This layer can include additional features like antimicrobial properties to minimize bacterial growth, further enhancing user comfort and hygiene.

In some non-limiting examples, the outer absorbent layer may be a hydrophilic, oleophilic, and/or amphiphilic layer. The outer absorbent layer may include cotton, cellulose, wool, nylon, rayon, PVA, PEG, polypropylene, polyethylene, polyester, polystyrene, silicone, surfactants, proteins, block copolymers, amphiphilic gels, microfibers, or a combination thereof, and can be treated to enhance hydrophilicity, oleophilicity, and/or amphiphilicity. One or more of the materials may be cosmetic grade.

Proximal the outer layer, the moisture-locking interior layer is responsible for retaining absorbed fluids, ensuring they do not reach the skin or cause the guard to become overly saturated. Made from advanced absorbent technologies, this layer may include superabsorbent polymers (SAP) and/or hydrophilic polyurethane foam, designed to hold moisture securely for long-lasting wear.

The moisture-locking interior layer may enable extended use by effectively capturing and retaining moisture. Its design prevents backflow or moisture transfer to the skin where moisture is meant to be dabbed off, as well as to clothing or other surfaces. This containment ensures that absorbed fluids stay securely locked, even under pressure or movement, maintaining the integrity of the makeup layer above.

In some non-limiting examples, the moisture-locking interior layer may be a hydrophilic, oleophilic, and/or amphiphilic layer. The moisture-locking interior layer may include cotton, cellulose, wool, nylon, rayon, PVA, PEG, polypropylene, polyethylene, polyester, polystyrene, silicone, surfactants, proteins, block copolymers, amphiphilic gels, microfibers, or a combination thereof, and can be treated to enhance hydrophilicity, oleophilicity, and/or amphiphilicity. Additionally or alternatively, the moisture-locking interior layer may include superabsorbent polymers (SAP), fluff pulp, nonwoven fabric, acquisition and distribution layer (ADL), hydrophilic, oleophilic, and/or amphiphilic polyurethane foam and can be treated to enhance hydrophilicity, oleophilicity, and/or amphiphilicity. One or more of the materials may be cosmetic grade.

The base adhesive layer of the guard may include a hypoallergenic, skin-safe adhesive designed to keep the guard securely in place on non-facial and non-sensitive areas such as fingers, knuckles, hands, wrists, forearms, and shoulder area with the purpose to dab moisture from the face and body. The guard is breathable, allowing for comfortable wear in high-movement areas and remaining stable even in humid or physically demanding environments. The base adhesive layer may be formed from flexible adhesives such as acrylic or silicone-based materials, and it may minimize skin irritation and does not leave residue upon removal. Additionally, the adhesive strength can be customized to accommodate different skin sensitivities and body locations. Optional micro-texturing and grip-enhancing patterns further improve adhesion, allowing the guard to maintain its position throughout use.

In some non-limiting examples, the base adhesive layer may be include a skin-friendly, flexible, breathable adhesives such as acrylic, silicone-based, hydrocolloid, polyurethane-based, hydrogel, collagen-based, or a combination thereof. One or more of the materials may be cosmetic grade.

The guard may further include a protective liner. The protective liner may be attached to, for example, the adhesive layer, and may include materials such as silicone-coated paper, polyethylene (PE) film, or wax-coated paper sufficient to maintain the adhesive's cleanliness and stickiness prior to application. This liner ensures that the guard remains sterile and uncontaminated, supporting hygiene standards for sensitive body areas.

In some non-limiting examples, the protective liner may include silicone-coated paper, polyethylene (PE) film, polypropylene (PP) film, wax-coated paper, polyvinyl chloride (PVC) film, fluoropolymer-coated paper, or a combination thereof.

According to some aspects, all or a portion of the guard may be free of active ingredients. As used herein, an “active ingredient” refers to a component that is biologically active and responsible for an intended biological effect. According to some aspects, one or more of the outer absorbent layer, the moisture-locking interior layer, the base adhesive layer, and the removable protective liner may be free of active ingredients as disclosed herein.

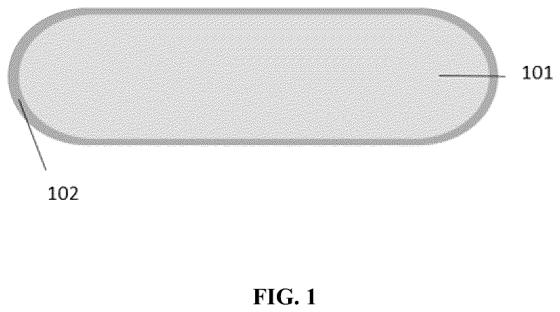

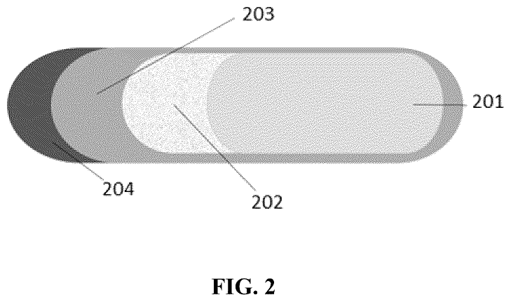

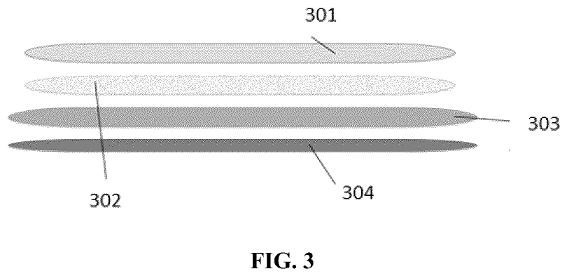

FIG. 1 shows an example plan/top view of a guard according to aspects of the present disclosure, including an outer absorbent layer 101 and a base adhesive layer 102, as described herein. FIG. 2 shows an example sectional view of a guard according to aspects of the present disclosure, including an outer absorbent layer 201, a moisture-locking interior layer 202, a base adhesive layer 203, and a removable protective liner 204. FIG. 3 shows an example exploded view of a guard according to aspects of the present disclosure, including an outer absorbent layer 301, a moisture-locking interior layer 302, a base adhesive layer 303, and a removable protective liner 304.

The guard optionally utilizes cosmetic-grade materials in key layers, particularly in the absorbent layer and adhesive layer, to ensure compatibility with makeup and skincare products. Cosmetic-grade materials provide a hypoallergenic, skin-friendly surface that minimizes the risk of smudging, caking, or disrupting applied makeup, even with frequent wiping. Additionally, these materials maintain the breathable quality needed for long-lasting wear, preventing discomfort and allowing the skin to remain fresh under high-moisture conditions. The cosmetic-grade options distinguish the guard from general-purpose absorbent products, offering unique compatibility with cosmetics to meet the needs of users in professional, social, and personal care settings where appearance preservation is essential.

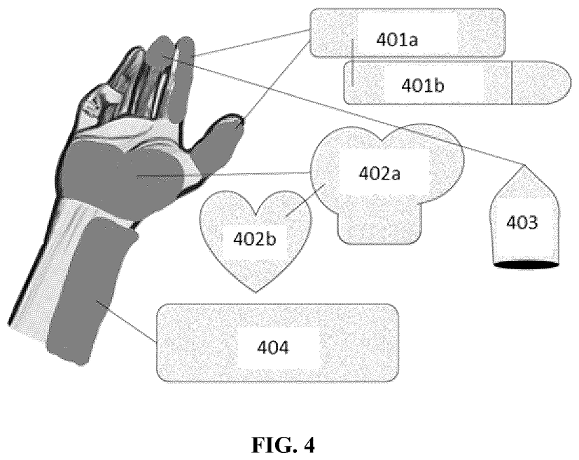

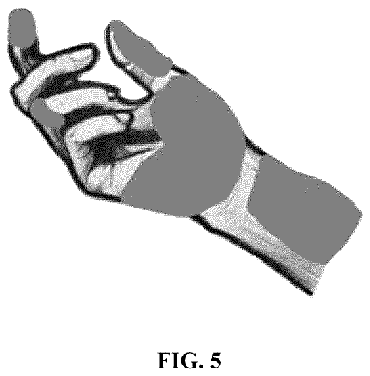

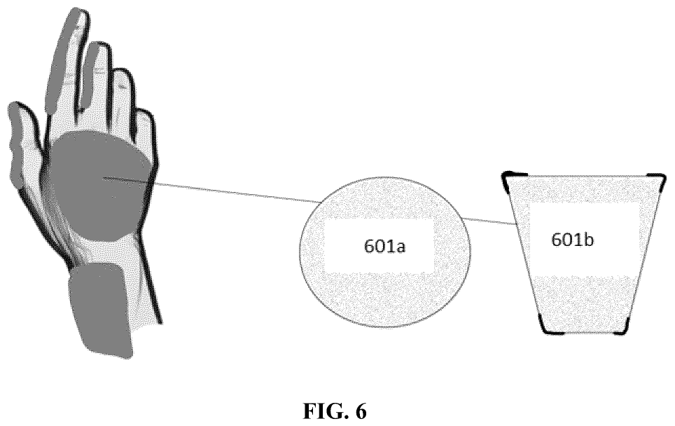

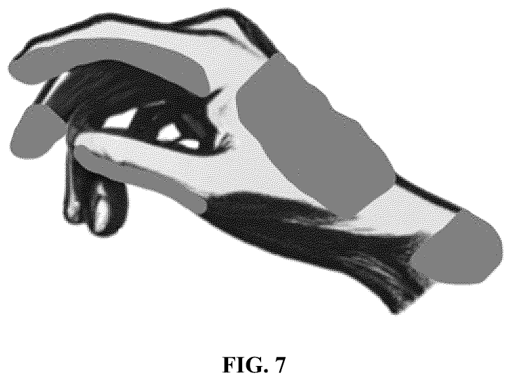

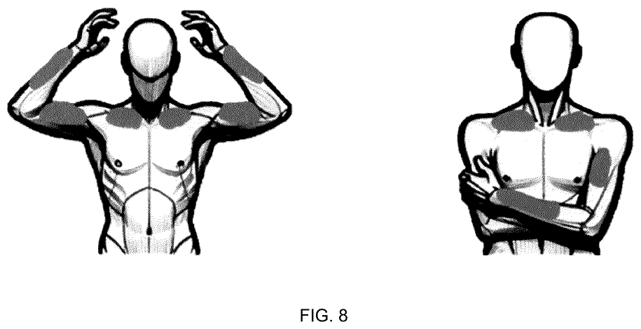

According to some aspects, the guard may be shaped for application to one or more body parts, including the fingers (including the tips of the fingers), knuckles, palms, back of hands, wrists, forearms, biceps, shoulders, back, chest, neck, or a combination thereof. FIG. 4 shows example guards of the present disclosure as worn by a user, including finger guards 401a and 401b, palmar guards 402a and 402b, a fingertip guard 403, and a wrist guard 404. FIGS. 5, 6, 7, and 8 show other example guards of the present disclosure as worn by a user. In particular, FIG. 6 shows example guards 601a and 601b which may be worn on the top of a hand.

In some non-limiting examples, the guard may be applied by to a user by removing the protective liner, positioning the guard, and applying the guard to one or more body parts via the base adhesive layer. A user may then discreetly dab moisture from the face or body by pressing the guard against areas where sweat, tears, or oils accumulate. To remove, a user may gently peel the guard away from the skin.

According to some aspects, the guard of the present disclosure may be paired with certain makeup application methods and products that extend wear, reduce oiliness, and/or enhance adherence. When used together, these complementary techniques create a cohesive makeup maintenance system, allowing the guard to work at its full potential with minimal makeup disruption.

Using a makeup primer before foundation application creates a smooth, adhesive base, which helps makeup stay in place. Primers designed to fill pores or mattify the skin add durability, reducing the likelihood of makeup transfer onto the guard. By securing the makeup to the skin, primer enhances the guard's ability to absorb moisture without disturbing the underlying makeup layer.

Product such as oil-control moisturizers or mattifying primers keep excess oils at bay, allowing the guard to focus on absorbing sweat and tears. For users with oily skin, these products reduce shine and prevent makeup slippage, ensuring that the guard's absorbent surface remains focused on moisture without risking smudging or makeup breakdown due to oil.

Waterproof or long-wear formulations in foundations, mascaras, and eyeliners are designed to withstand moisture exposure. This durability aligns with the guard's functionality by reducing the risk of smudging or running, ensuring that the guard can absorb sweat and tears without disturbing or streaking the makeup. This pairing is especially effective for events or activities where long-lasting makeup is desired.

Setting sprays add a layer of protection, locking makeup in place, while fixing sprays provide additional reinforcement for extended durability. When makeup is set, it becomes more resilient to smudging and transfer, allowing the guard to absorb moisture without affecting the makeup layer underneath. This pairing reduces the likelihood of streaks or disruptions, making it ideal for users who need reliable, long-lasting results.

Setting powder, especially in a matte or translucent form, locks in foundation and controls shine. The powder creates a soft barrier, minimizing the impact of sweat or tears and making it easier for the guard to perform without makeup transfer. This additional step enables the guard to absorb moisture effectively, preserving a fresh, matte finish.

Thinner, lightweight makeup layers adhere more effectively to the skin and are less prone to streaking. By applying makeup in light, buildable layers, users can enhance the stability of their makeup, allowing the guard to absorb moisture without affecting makeup layers. This approach is particularly helpful for users who need to dab frequently, ensuring their makeup remains intact.

The guard's multi-layer design is engineered to withstand high levels of moisture, providing durable moisture control while preserving the integrity of applied makeup or skincare. The outer absorbent layer wicks moisture away from the skin, channeling it to the moisture-locking interior layer, which securely retains fluids without allowing re-contact with the skin. The base adhesive layer ensures a secure hold, even in humid or high-movement conditions, while maintaining comfort and breathability. This layer interaction allows the guard to perform reliably in intense environments, such as long work shifts, high-energy athletic activities, or outdoor events, ensuring both functionality and comfort for the user.

The guard's multi-layer structure is engineered to provide comprehensive moisture management through the complementary functions of each layer, particularly under sustained use in high-moisture environments. The outer absorbent layer wicks moisture away from the skin, drawing it toward the moisture-locking interior layer, which captures and securely retains the absorbed fluids. This layered interaction prevents re-contact with the skin, ensuring that moisture remains contained within the guard without compromising makeup or skincare applied to the body.

The breathable base adhesive layer reinforces the guard's stability and comfort, allowing it to remain securely adhered to the skin even in humid, high-activity conditions. Together, these layers provide a continuous and balanced system that maintains a dry surface against the skin, supporting long-lasting wear without requiring frequent adjustments or replacements. This interaction ensures that the guard effectively manages sweat, tears, or other moisture over long periods, making it ideal for use during lengthy events, outdoor activities, and physically demanding situations.

The guard's multi-layer structure is designed to operate synergistically, allowing each layer to enhance and support the functions of the others, particularly under prolonged and intense moisture exposure. For instance, during high-activity events such as athletic activities or outdoor performances, the outer absorbent layer actively wicks sweat or tears away from the skin, channeling it efficiently to the moisture-locking interior layer. This interior layer is engineered to retain absorbed moisture securely, preventing it from migrating back to the surface and ensuring a dry, comfortable feel against the skin.

The base adhesive layer complements this moisture-management process by providing a breathable yet secure hold, designed to remain flexible and stable despite environmental challenges like humidity or constant movement. As the guard absorbs more moisture, the adhesive layer's breathability allows for continuous airflow, preventing skin irritation and maintaining a comfortable fit throughout prolonged wear.

In situations such as long work shifts or events like weddings, where durability and comfort are essential, this layer interaction provides users with reliable, long-lasting protection. Each layer's contribution to moisture-wicking, retention, and secure adhesion ensures that the guard performs consistently across various conditions, making it ideal for extended use without the need for frequent adjustments or replacement.

The present disclosure is also directed to methods of using the guard described herein. For example, the guard may be used in a method of discrete tear management. Individuals often use subtle techniques to prevent tears from smudging makeup or drawing attention, such as pressing the tear duct, tilting the head, or discreetly dabbing with fingers. However, these methods are not always effective or convenient. The guard provides an improved, discreet, and efficient solution that optimizes each of these approaches by providing a specialized absorbent surface designed specifically for moisture management without compromising makeup or hygiene.

A method of discrete tear management as described herein may include one or more of the following.

### Pressing the Tear Duct

Many people discreetly press their tear ducts—the inner corners of the eyes—to prevent tears from spilling onto their cheeks. This approach, however, only absorbs a limited amount of moisture and requires careful control to avoid disturbing makeup. The guard, designed to adhere comfortably on the inner finger or palm, provides an absorbent, hygienic surface for pressing the tear duct. By allowing users to absorb tears directly at the source, it prevents moisture from reaching the cheeks without disturbing makeup. Additionally, the guard offers a controlled absorption capacity, making it more reliable and effective than using bare fingers, which can introduce oils or bacteria to the skin.

### Tilting the Head

In social or professional settings, individuals sometimes tilt their heads slightly to slow tears from spilling over. This method, while effective to an extent, is not always practical or discreet and may inadvertently draw attention. The guard allows users to discreetly catch and absorb tears without requiring head movements, maintaining a natural posture and reducing the need for visible adjustments. With the guard positioned on a finger or the palm, users can subtly dab tears away without compromising their natural composure, providing an immediate and reliable solution.

### Discreet Dabbing With Fingers

When tear management requires dabbing, individuals often use their fingers to gently dab away moisture. However, this technique can lead to makeup disruption, oil transfer, and even the potential spread of bacteria. The guard mitigates these issues by offering a dedicated, absorbent surface that captures tears while protecting makeup. Unlike fingers, which can transfer natural oils and contaminants, the guard is composed of materials specifically chosen to absorb moisture efficiently while maintaining the integrity of any cosmetics. This not only preserves appearance but also ensures hygiene, providing a practical solution that allows users to manage tears without concerns of smudging or infection.

### Discreet Dabbing With Knuckles

Many individuals instinctively use their knuckles to dab away tears in a discreet, natural manner. This method allows for a subtle and efficient way to manage tears without relying on a tissue or other object, as the knuckles naturally reach the eye area with minimal motion. The guard's design accommodates this common gesture by providing a dedicated absorbent surface on the knuckles, ensuring effective moisture capture while maintaining a clean, dry surface that doesn't disturb makeup. By using the guard on the knuckles, users can discreetly manage moisture without the risks of transferring oils or smudging cosmetics.

### Discreet Dabbing With the Inside of the Palm

In situations where subtlety is paramount, individuals may choose to discreetly press the inside of their palm to the face, front or nape of neck, front or lower hairline at nape of neck, or anywhere on the body to absorb moisture. This natural gesture provides an inconspicuous way to manage tears or sweat without obvious hand movements near the face. The guard, designed for placement on the inner palm, offers a targeted absorbent surface that discreetly captures moisture while preventing transfer to other areas. This approach allows for effective moisture control without drawing attention, preserving makeup integrity and ensuring hygiene. By utilizing the inner palm, users benefit from a larger absorbent surface area, enabling quick and seamless tear management.

According to some aspects, the guard includes quick-absorbing properties for maintaining the integrity of makeup and topicals. Sweat, tears, and oils, particularly when combined with body heat, can accelerate the breakdown of makeup products by disrupting the emulsifiers and binding agents that keep the products intact.

The rapid absorbency of the outer absorbent layer of the guard ensures that moisture is immediately captured upon contact, preventing the prolonged exposure of makeup and topicals to both moisture and heat. By doing so, the guard prevents the heat from sweat and tears from chemically breaking down makeup and topicals. This mechanism stops emulsification, where water and oil-based makeup products separate, causing them to smudge, fade, or degrade.

Makeup and topicals often contain emulsifiers that keep oil and water phases blended together. When excess moisture comes into contact with these products, it can destabilize the emulsifiers, causing the makeup or topicals to break down. Similarly, body heat can speed up these chemical reactions, leading to quicker degradation.

The guard's ability to absorb and lock in moisture ensures that these fluids do not interfere with the applied products to the face and the body, thereby preserving the makeup's structure and chemical composition. By reducing exposure to moisture, the guard also prevents smudging, streaking, or fading that commonly occurs in high-moisture environments such as weddings, outdoor events, or during physical activities.

Sweat contains both water and salts, which can degrade makeup over time. Additionally, facial oils can cause oil-based products to become greasy or uneven. The guard's oil-absorbing properties help to manage these oils before they interfere with the makeup's texture and appearance.

The rapid absorption of both sweat and oils allows the user to maintain a flawless appearance, as the guard actively prevents moisture from disrupting the makeup or topicals applied to the face and body.

The guard of the present disclosure may be designed for application to areas of the body such as the fingers, knuckles, hands, wrists, forearms, biceps, and shoulder area for discreet and efficient use. For example, it can be applied/adhered to the inside or outside of the index finger or wrist, allowing the user to naturally, comfortably, and discreetly dab away moisture from the face or body without drawing attention. The guard's adhesive properties ensure it stays in place, providing long-lasting protection.

By strategically positioning the guard in areas where moisture can be discreetly dabbed off, the user can prevent the breakdown of makeup and topicals, ensuring they stay intact for long periods, even in high-moisture environments.

The guard of the present disclosure may include one or more of the following functional characteristics.

The outer absorbent layer may be engineered to retain moisture without becoming oversaturated. This design makes the guard suitable for high-moisture and high-activity environments, including physical performances, athletic events, and extended shifts in food service or hospitality. The guard's multi-layer structure ensures that moisture is quickly absorbed and securely retained, preventing makeup from smudging or fading due to exposure to fluids.

The guard may be designed with a highly adaptable moisture intake layer capable of absorbing a variable range of liquid volumes, adjusted based on the guard's size and thickness. Smaller, thinner guards are optimized for moderate moisture intake, suitable for absorbing lighter volumes of liquid in the range of milliliters, ideal for low-level perspiration or brief exposure. Larger, thicker guards, however, are structured to manage significantly higher volumes, allowing for high-moisture environments or prolonged use, with the capacity to intake greater quantities in the range of hundreds of milliliters or more, such as up to 3 liters.

The guard may include thermal or cooling properties, providing a refreshing effect on the skin during use. This feature enhances comfort in hot environments or during physical exertion, reducing discomfort while supporting makeup integrity.

The guard may be available in a diverse range of precut or customizable shapes (with options in both two-dimensional and three-dimensional), each designed to meet specific user needs while minimizing peeling, snagging, or lifting from the skin. Shapes such as rounded, oval, teardrop, hexagonal with softened corners, butterfly, hourglass, square, and rectangular provide ergonomic fits tailored to different body areas, such as fingers, knuckles, wrists, forearms, and clavicles. These shapes offer enhanced adhesion and stability by conforming closely to the body's natural contours, ensuring discreet and secure placement during high-movement activities or prolonged wear.

Rounded shapes or those with softened corners, such as hexagonal or teardrop, distribute stress more evenly and are less likely to catch on clothing or other surfaces. This reduces the likelihood of lifting or peeling at the edges, which is why these shapes are often used in adhesives and wearables that need to stay secure on curved surfaces, such as the skin.

Certain shapes, like butterfly and hourglass, provide more surface area, which can enhance adherence and stability, especially on larger or more mobile body parts (e.g., wrists, forearms). More coverage helps spread the adhesive's force over a broader area, reducing the chance of peeling or lifting from movement.

Shapes with softer contours, like oval or rounded designs, reduce the number of contact points along the edges that could catch or lift, which is beneficial in wearables designed for flexible body areas.

The guard's multi-layer structure may designed to maximize moisture control and comfort across various customizable shapes. For example, the outer absorbent layer, constructed from microporous, breathable materials, may enhances moisture-wicking efficiency while conforming to ergonomic shapes like rounded, teardrop, and hexagonal with softened edges. In some non-limiting examples, the outer absorbent layer may include a network of interconnected pores. In some non-limiting examples, the network of interconnected pores may be distributed uniformly across the surface. According to some aspects, all or a portion of the pores may have a diameter of between about 5 mm and about 100 mm, ensuring effective breathability and moisture management. The textured surface is configured to provide a soft, comfortable contact with the skin, reducing irritation and enhancing user comfort during prolonged use. This specific texture is integral to the functionality of the absorbent guard, ensuring that the product maintains its micrometers, ensuring effective breathability and moisture management. The porous structure enhances the material's absorbency, allowing it to quickly wick moisture away from the surface of the skin and distribute it throughout the material. The outer absorbent layer's material is a soft and gentle texture, making it suitable for direct contact with the skin. This layer may include a plurality of raised and recessed regions arranged in a precise pattern to improve its functional properties. This layer may reduce the likelihood of peeling or lifting, particularly in high-movement areas such as knuckles or wrists, where the skin flexes with motion.

Each layer may be engineered to support the guard's secure and discreet placement on targeted areas. For example, the interior moisture-locking interior layer may provide enhanced stability for larger shapes, like rectangular or butterfly configurations, used on forearms or clavicles, ensuring durability even in prolonged, high-moisture settings. Meanwhile, the base adhesive layer may provide skin-safe, hypoallergenic adhesion, adapting seamlessly to shapes that are more compact and require flexibility, such as those applied to finger joints or smaller skin surfaces.

The is integration of layer-specific benefits with unique shapes and placement areas enhances the guard's overall functionality, allowing users to select options that balance adherence, comfort, and moisture management for optimal performance in various real-world applications.

The guard can be offered in multiple sizes, ranging from extra small to 3XL, with thickness options adjustable for various absorbency needs. Users can select from ultra-thin layers for minimal visibility or thicker layers for prolonged absorbency in high-moisture settings.

In some non-limiting examples, the outer absorbent layer may have a thickness of between about 0.01 inches and 2.5 inches. In some non-limiting examples, the moisture-locking interior layer may have a thickness of between about 0.01 inches and about 2.5 inches. In some non-limiting examples, the base adhesive layer may have a thickness of between about 0.01 inches and about 0.5 inches.

In some non-limiting examples, each layer of the device may independently have at least one dimension that is between about 0.5 and 24 inches, such as a width, length, diameter, or combination thereof.

For optimal discretion or visibility, the guard may be available in a spectrum of colors of the rainbow, from skin tones and neutrals to bold hues, clear, and translucent options, artistic designs, or images. Users may also choose from a variety of textures, including smooth, micro-textured, grid, dotted, or organic wave designs, each enhancing either discreet wear or improved tactile control for dab motions. These textural choices serve both aesthetic and functional purposes, ensuring user comfort and satisfaction in both public and private settings.

Constructed from environmentally friendly, eco-friendly, biodegradable, and/or recyclable materials, the guard supports sustainability goals. Designed as a single-use product, the guard provides high performance while minimizing environmental impact. Each guard is individually packaged in recyclable, sterile containers to maintain hygiene.

The guard is designed for long-lasting wear, remaining effective for up to 10 hours in high-moisture conditions. It provides consistent adherence and moisture protection during prolonged events, such as weddings, sports activities, and all-day conferences. The durable adhesive layer ensures that the guard stays securely in place, even during active movement, without requiring constant adjustment or replacement.

The adhesive and absorbent materials may be hypoallergenic and/or dermatologically tested, ensuring that the guard is compatible with sensitive skin. The guard minimizes the risk of irritation, providing a comfortable and safe experience for prolonged wear. Unlike conventional tissues, the guard's skin-safe, breathable adhesive supports long-lasting use without discomfort.

The guard may feature a cut-to-fit design, allowing users to trim the guard into custom shapes and sizes, providing a tailored fit for unique application areas. This design empowers users to modify the guard to meet specific needs, whether for precise coverage on smaller areas like knuckles and fingers or for creating broader, custom-sized sections for larger areas like the wrist, forearm, or clavicle.

The guard's layered structure maintains its integrity and adhesive properties even after being trimmed, ensuring consistent moisture absorption, adherence, and comfort regardless of size alterations. This flexibility offers users a high degree of personalization, making the guard suitable for a variety of body areas, activities, and moisture control needs, from discreet single-use applications to versatile coverage in high-moisture settings.

The guard is available in a range of shapes, such as rounded, oval, teardrop, hexagonal with softened corners, butterfly, hourglass, square, and rectangular configurations, each specifically designed to minimize peeling, snagging, or lifting from the skin. These shapes allow the guard to blend seamlessly with natural body contours, reducing visibility for a discreet appearance. The optimized perimeter provides enhanced adhesion and wearability, ensuring a secure fit even in high-movement areas, while maintaining comfort and aesthetic discretion for daily activities and public settings.

The guard may be designed with a modular, multi-part system that allows users to combine and arrange multiple components to customize moisture control based on specific needs and application areas. Each module may attach seamlessly to others, enabling users to configure the guard for small, focused areas, such as knuckles or fingertips, or to layer modules for broader coverage on larger areas, like the forearm or shoulder. This customizable system provides users with flexibility to adjust absorbency levels by layering modules with different thicknesses or absorbent capacities, offering adaptable solutions for various moisture conditions, such as athletic events, outdoor settings, or prolonged wear during public engagements.

The guard may be available in reusable designs, with durable materials that allow it to be washed, sanitized, and stored for multiple uses while maintaining hygiene.

A feature that visually signals the guard's moisture saturation, such as a color-changing layer, may alert the user when the guard has reached its maximum absorbency.

In addition or as an alternative to the base adhesive layer, the guard may include non-adhesive options using gentle compression, elastic bands, or adjustable loops to provide secure attachment without direct skin contact, suitable for users with adhesive sensitivities.

Versions featuring perforations or breathable mesh sections for increased airflow and comfort, particularly for use in warm or high-activity conditions.

Interchangeable or adjustable layers allow users to choose specific thicknesses, textures, or absorbency levels for various needs, such as thicker layers for long-lasting wear.

The guard may be available with infused scents or essential oils for a refreshing, calming experience, providing a subtle fragrance while maintaining its primary function.

A hypoallergenic or child-safe design offers ultra-gentle materials, allowing safe use for sensitive skin or younger users.

A reinforced design for use in high-movement or high-sweat conditions, featuring stronger adhesives or thicker layers for long-lasting performance.

Packs that include guards in various sizes, thicknesses, and absorbency levels for versatility in changing conditions or user preferences.

The guard may fold or compress easily, allowing for discreet portability in small spaces such as pockets or small bags.

Both adhesive-based and non-adhesive options may be available, offering flexibility for users who prefer either skin-contact or hands-free application.

Integration of a small sensor may be provide in order to notify users when the guard reaches maximum absorbency, with optional connectivity to a smartphone app for alerts.

Guards designed from biodegradable or compostable materials, catering to users with environmental sustainability preferences.

Options with functional surface patterns, such as gentle ridges for massage-like effects, may be provided to offer additional sensory benefits.

Anti-microbial or anti-bacterial properties may be provided to reduce bacterial growth on the surface, especially for long-lasting wear applications.

An expandable or stretchable material maybe provided that conforms to a variety of body types and sizes, enhancing comfort and usability.

A premium version with high-quality finishes, decorative elements, or limited-edition packaging for luxury or special occasion markets may be provided.

Channels or grooves that direct moisture toward highly absorbent areas, enhancing absorption efficiency and preventing saturation, may be provided.

A two-sided guard with varied textures on each side, offering both a soft and textured option for user flexibility, may be provided.

Replaceable adhesive strips for users who need flexibility in placement, extending the guard's usability without compromising adherence, may be provided.

A coating that prevents static, ensuring the guard does not disturb or interfere with makeup during application, may be provided.

Gentle compression points that provide a massage-like effect, offering relaxation benefits during use, may be provided.

Guards available in colors that blend with common clothing shades, such as black or beige, enhancing discretion when used on exposed areas, may be provided.

Removable tabs for hands-free removal, maintaining hygiene standards in food service or healthcare environments, may be provided.

Seasonal or themed designs, such as holiday or limited-edition styles, to add a personal or collectible element, may be provided.

Adhesives that strengthen with body heat, ensuring a secure hold in warm environments or during physical activity, may be provided.

A cooling layer that activates upon contact with moisture, providing a refreshing effect during intense activities, may be provided.

An outer layer that “recharges” by exposure to air, allowing it to regain absorbency for multiple uses within a single wear, may be provided.

Ultra-gentle materials for users with sensitive or allergy-prone skin, enhancing comfort and usability, may be provided.

A set including additional accessories, like a mini mirror, setting spray, or compact storage, to complete the user's makeup maintenance kit, may be provided.

A refillable option where users replace the inner layers but keep a reusable outer layer, reducing waste and enhancing eco-friendliness, may be provided.

Adhesive that becomes more effective as it detects moisture, keeping the guard in place during high-moisture events, may be provided.

Guards that can adjust in shape (e.g., oval to rectangular), ensuring comfortable and versatile use on different areas, may be provided.

A specialized version designed for post-surgical moisture management, suitable for sensitive or healing skin, may be provided.

QR codes or packaging with interactive content, such as tutorials, usage tips, or community support for users, may be provided.

A subscription service that delivers guards tailored to users' activities and preferences, providing convenience and ensuring regular supply, may be provided.

A quick-drying outer layer that retains absorbency for multiple dabs in one use, preventing saturation over time, may be provided.

Stackable layers allowing users to customize thickness or texture for various applications, from lightweight daily use to thick, long-term wear, may be provided.

Color-changing features that indicate temperature changes, helping users anticipate moisture buildup before it affects makeup, may be provided.

Optional biodegradable clips to secure the guard on larger areas, enhancing durability during high-movement activities, may be provided.

Ergonomic designs that match specific body parts like wrists or knuckles, enhancing comfort and effectiveness, may be provided.

Versions with reflective or glow-in-the-dark properties for nighttime or low-light environments, may be provided.

Fragrance-releasing properties activated by moisture, adding a subtle scent each time the guard absorbs sweat or tears, may be provided.

A guard with layers that progressively absorb moisture, allowing users to peel away saturated outer layers for long-lasting use, may be provided.

A durable, travel-friendly case with optional UV-light sanitation for hygiene, especially for reusable or multipurpose versions, may be provided.

A thin, gel-based layer integrated into the guard to wick away moisture even faster, providing additional protection while offering a cooling, cushioned effect on the skin, enhancing comfort for long-lasting wear, may be provided.

Guards designed with flexible bends and contoured shapes that comfortably adhere to joints, such as knuckles and finger creases, allowing users to maintain full motion without sacrificing fit or comfort, may be provided.

A microporous layer that promotes airflow through the guard, enabling faster drying and reducing dampness, making it ideal for high-moisture environments or active wear, may be provided.

Subtle textured grips along non-absorbent edges to ensure the guard remains securely in place, even when applied to smooth or high-movement skin areas, enhancing stability during physical activities, may be provided.

An anti-irritant coating that adds an extra layer of protection for highly reactive or allergy-prone skin, creating a barrier that minimizes the risk of irritation from prolonged wear, may be provided.

Integrated stretch fabric that moves naturally with the skin, providing a snug and flexible fit on high-movement areas like fingers, knuckles, and wrists without slipping or detaching, may be provided.

Elastic fasteners or bands added to non-adhesive versions, offering a secure hold on wrists, forearms, or other areas, particularly beneficial for athletic or high-activity settings, may be provided.

A scent-neutralizing layer that absorbs unwanted odors, making the guard an ideal choice for high-sweat situations such as exercise, outdoor events, or long work shifts, may be provided.

Peel-off tabs or strips for easy application and removal, reducing direct handling of the adhesive and keeping the guard clean and ready for use, particularly beneficial for hygiene, may be provided.

Reflective edges or strips integrated into the guard, increasing visibility in low-light environments, ideal for nighttime events, outdoor activities, or high-visibility needs, may be provided.

Small, attachable heat or cooling pads that can be added to the guard for additional warmth or a cooling effect, enhancing user comfort based on preference or environmental conditions, may be provided.

An ultra-thin, nearly invisible clear version that blends seamlessly with the skin, offering a highly discreet option for users prioritizing subtlety and a “barely there” appearance, may be provided.

A biodegradable adhesive option that fully decomposes, making the guard and its adhesive eco-friendly, contributing to sustainable practices and reducing environmental impact, may be provided.

Stackable guards that can be layered for increased absorbency, allowing users to adjust the guard's capacity based on anticipated activity levels or moisture needs, may be provided.

Guards designed with an integrated sleeve or pocket to hold them in place, especially useful on wrists, forearms, or other high-movement areas for enhanced security and comfort, may be provided.

Guards with varying absorbency zones, such as high-absorbency edges, tailored to control moisture precisely based on placement, optimizing performance for each body area, may be provided.

A suction-based adherence mechanism for secure but temporary attachment on surfaces where adhesive may not be ideal, allowing easy reapplication and reuse, may be provided.

An innovative multi-layer absorbency design that maximizes moisture retention in key areas prone to exposure, reducing overall bulk while maintaining peak functionality, may be provided.

A companion smartphone app that connects to the guard, particularly useful if the guard has sensors, tracking usage duration, moisture levels, and providing reminders for replacement, may be provided.

Customization options with monograms or engravings for users desiring a personalized touch, adding uniqueness and an individualized feel to each guard, may be provided.

The guard of the present application may provide specialized application and targeted use

The guard's design enables discreet, efficient moisture absorption without resembling traditional tissue products. Positioned on non-facial areas, the guard facilitates subtle dabbing or wiping motions, allowing users to manage sweat, tears, or oils naturally and inconspicuously. This feature is especially beneficial in professional or social settings where appearances matter, such as weddings or public performances.

Intended for diverse high-moisture settings, the guard is particularly valuable for cosmetics, sports/athletics, and food service industries, where long-lasting makeup preservation is essential. By effectively absorbing moisture on hands, wrists, and other non-sensitive areas, the guard prevents cosmetic degradation across different environments and climates, offering versatility in professional and personal settings.

Packaged in a sterile, resealable, and recyclable container, the guard remains hygienic and convenient for single-use or possible reusable application. The packaging is designed to ensure ease of access while preserving the cleanliness of each guard, aligning with the unique hygiene requirements for makeup preservation products.

The guard may optionally include an absorbency indicator to signal when it has reached its maximum saturation capacity. This indicator, designed as a potential visual cue, allows users to identify when the guard requires replacement to maintain optimal functionality. While not essential to all embodiments, the indicator feature supports continuous protection in scenarios that benefit from prolonged wear, such as events or environments where users may experience ongoing moisture exposure. The inclusion of an indicator enhances user awareness and convenience, offering added value for use of guard.

Additional optional features may include fragrance or scent-blocking properties, which neutralize odors or add a pleasant scent, enhancing user experience during prolonged wear. The guard may also be available as a multi-part system, allowing users to combine components for customized moisture absorption and cosmetic protection tailored to specific needs.

This disclosure ensures makeup and topicals applied to the face and body remain intact in high-moisture environments, while also providing significant cost savings for consumers by reducing the need for excessive makeup, frequent touch-ups, and professional makeup artist services. Some key use cases include:

Weddings and Special Events: Brides, bridal parties, and guests often need long-lasting makeup through ceremonies, photoshoots, and receptions. The guard's ability to maintain makeup integrity eliminates the need for costly touch-ups and professional makeup services throughout the event, reducing expenses for clients and event planners alike.

Long Work Shifts in Hospitality and Food Service: Professionals like chefs, waitstaff, and bartenders frequently encounter high humidity and heat during long shifts. The guard maintains makeup for these workers, minimizing makeup reapplication and decreasing costs associated with product purchases and on-site makeup artist services.

Athletic and Performance Events: Athletes, dancers, and performers can rely on the guard to maintain their appearance during physically demanding events. This protection helps reduce the need for reapplication and the associated expenses of on-set makeup services.

Conferences and Public Speaking: For professionals presenting at all-day conferences, the guard ensures a polished look without requiring reapplications. This prevents additional spending on makeup services for high-stakes public engagements.

Travel: Travelers, particularly business professionals, face prolonged transit and challenging environments that degrade makeup. The guard preserves makeup quality, reducing the need for multiple products and minimizing expenses related to frequent reapplication and portable makeup kits.

The moisture-absorbing guard may provide consumers with significant savings in both cost and time by reducing the need for frequent restroom breaks to reapply makeup. By effectively managing sweat, tears, and oils, the guard helps maintain makeup throughout the day, cutting down on the frequency and amount of product needed for touch-ups. This feature is especially beneficial for those using premium cosmetics, as it helps extend product life without requiring repeated reapplications. Additionally, the guard's durability lessens the need for professional touch-ups during long events, such as weddings or conferences, where maintaining a polished look is crucial. With fewer restroom visits and touch-ups, the guard offers a convenient, budget-friendly solution for makeup preservation in high-moisture situations.

While safety concerns may arise regarding potential bacteria growth or irritation from moisture-absorbing products, the guard is designed to mitigate these risks effectively. Many everyday products used in makeup and body care, such as mascara, sweat rags, and false eyelash adhesives, are less safe in comparison. For instance, mascara can run or smudge when exposed to tears or sweat, potentially causing eye irritation or even infections. Sweat rags and towels, frequently reused, are prone to bacterial buildup, while eyelash adhesives can degrade with moisture, leading to allergic reactions or skin irritation. Unlike these products, the moisture-absorbing guard is single-use, hypoallergenic, and designed to absorb moisture directly upon contact, providing a safer, more sterile alternative for managing moisture while preserving makeup integrity.

Most dermatologists recommend wearing makeup for 8 to 12 hours to maintain skin health, as long-lasting wear can clog pores, cause irritation, and interfere with the skin's natural renewal process. Makeup that stays on the skin too long can trap oils, sweat, and environmental particles, potentially leading to breakouts and dryness. Products designed for long-lasting wear are generally formulated to last this long, but proper removal within this period is essential to keep skin clear and healthy.

The guard is optimized to align with these recommendations, designed to absorb moisture efficiently while being worn for up to, in one non-limiting examples, 10 hours. This feature allows users to maintain makeup integrity throughout a full day without exceeding safe wear times. The guard's high absorbency capacity ensures it can handle moisture buildup from sweat and tears without needing to be frequently replaced, minimizing disruption to makeup while keeping skin comfortable. By offering long-lasting wear with robust absorbency, the guard provides a practical solution that supports makeup longevity while adhering to skincare best practices.

This disclosure offers numerous advantages over traditional tissues for managing tears, sweat, and facial oils, particularly in situations where maintaining a flawless makeup appearance is essential. While tissues are commonly used to blot away moisture, they present several drawbacks compared to the convenience and efficiency of the guard, which is applied/adhered directly to clean, product-free fingers, knuckles, wrist, forearm, bicep and shoulder area. The following are key benefits that make this disclosure a superior alternative to traditional tissues.

The guard is applied/adhered to clean, product-free, non-facial, non-sensitive areas such as the fingers, knuckles, wrists, forearms, biceps, and shoulder area, leaving the user's hands free to handle multiple tasks without the need to constantly reach for a tissue. This is especially beneficial in situations where individuals are multitasking, such as holding a bouquet at a wedding, managing cooking utensils in a restaurant, or participating in athletic activities. The guard ensures that users can simply discreetly dab away tears, facial oils or sweat without interrupting what they're doing or worrying about disrupting their makeup.

With tissues, individuals often need to carefully fold them to avoid smudging their makeup while discreetly dabbing or wiping away moisture. This process can be tedious and distracting, especially when emotions run high or during physically demanding activities. The guard eliminates the need for meticulous folding as it is already securely positioned on the fingers, hand, wrist, or body (not including face), ready to absorb moisture quickly and effectively, making it far more convenient and efficient.

Traditional tissues often leave behind small fibers or debris on the skin, especially after multiple uses. They can become soggy when saturated with tears or sweat, causing pieces to break off and cling to the face. The guard, on the other hand, does not disintegrate or leave residue on the skin. Its multi-layer design ensures that it absorbs moisture without breaking down or causing makeup to appear patchy or uneven.

Unlike tissues, which must be pulled out and held up to the face, the guard offers a much more discreet solution. Applied/adhered to areas such as the fingers, knuckles, wrist, forearm, bicep and shoulder area, it allows users to subtly and quickly manage sweat or tears without drawing attention to themselves. This is especially important in moments where appearances matter, such as at weddings, public events, or performances. Users can focus on the moment while keeping their makeup flawless, and their hands and artistic nails remain visible and free from holding a tissue.

Tissues typically require frequent replacement after becoming saturated with moisture, forcing users to constantly pull out new ones during high-moisture situations. This disclosure provides long-lasting, continuous use without the need for constant replacement. Its absorbent layers are designed to manage multiple instances of moisture throughout the day, offering long-lasting protection and convenience, making it much more efficient than traditional tissues.

Whether it's during an emotional wedding ceremony, a high-stakes business presentation, or a sports competition, this disclosure allows individuals to stay focused on the moment. Instead of fidgeting with tissues or worrying about their makeup smudging, users can discreetly dab away moisture and remain present in the event. The guard enhances the wearer's confidence by ensuring their makeup remains intact, allowing them to fully engage in the experience.

Traditional tissues, especially when folded or pressed against the face, can disturb makeup, causing foundation or powder to smudge or fade. The guard is specifically designed to absorb moisture without compromising the integrity of the makeup. Its non-abrasive surface allows users to blot sweat or tears without risking any disruption to their appearance.

Using tissues can be distracting in professional or personal settings, requiring the user to repeatedly reach into their pocket or bag to grab a new one. This disclosure is far more convenient and discreet, as it remains securely applied/adhered to areas such as the fingers, knuckles, wrists, forearms, biceps, and shoulder area and is ready for use whenever needed. Moreover, tissues often create unnecessary waste, especially during emotional events or physically demanding activities where multiple tissues may be needed. The guard reduces this waste, offering a more sustainable solution for moisture management.

High-quality makeup products can be expensive, and frequent reapplication due to smudging or makeup breakdown can be costly. Tissues, when overused, can cause makeup to wear off prematurely, leading to the need for touch-ups. The guard preserves makeup longevity, ensuring that cosmetics stay intact throughout the day without the need for constant touch-ups. This means fewer touch-ups and less product waste, leading to cost savings over time.

This disclosure represents a revolutionary change in how people manage tears, sweat, and facial oils. It replaces the outdated need for tissues in high-moisture situations, offering a more efficient, discreet, and cost-effective alternative. The guard transforms the way individuals protect their appearance, providing continuous protection and allowing users to focus on what truly matters without interruption.

The multi-layer, moisture-absorbing guard provides an innovative and efficient solution for maintaining the integrity of applied makeup and skincare topicals in high-moisture environments. By discreetly adhering to non-sensitive areas of the body, the guard allows users to dab away moisture from the face and body, preventing sweat, tears, and oils from disrupting cosmetics. Its advanced three-layer construction-comprising an outer absorbent layer, a moisture-locking middle layer, and a hypoallergenic adhesive base-ensures reliable moisture management throughout prolonged use.

This disclosure is particularly beneficial in high-moisture scenarios such as weddings, performances, and extended work shifts, providing a user-friendly, hands-free solution that minimizes the need for constant touch-ups. By addressing common issues in traditional products, such as smudging, contamination, and waste, the guard offers a hygienic, cost-effective option for individuals seeking long-lasting makeup preservation. With its customizable design, environmental responsibility, and versatility across personal and professional applications, the guard demonstrates a strong case for patentability, underscoring its unique and practical contributions to the beauty and personal care industries.

1. A moisture-absorbing guard comprising:
an outer absorbent layer comprising a network of interconnected pores,
a base adhesive layer, and
a moisture-locking interior layer between the outer absorbent layer and the base adhesive layer, wherein the moisture-locking interior layer comprises an absorbent material.

2. The moisture-absorbing guard of claim 1, wherein the moisture-absorbing guard is sized to be applied to fingers, hands, wrists, forearms, biceps, or a shoulder area.

3. The moisture-absorbing guard of claim 1, wherein the absorbent material comprises one or more superabsorbent polymers and/or a hydrophilic polyurethane foam.

4. The moisture-absorbing guard of claim 1, wherein the absorbent material comprises cotton, cellulose, wool, nylon, rayon, PVA, PEG, polypropylene, polyethylene, polyester, polystyrene, silicone, surfactants, proteins, block copolymers, amphiphilic gels, microfibers, a fluff pulp, a nonwoven fabric, an acquisition and distribution layer, or a combination thereof.

5. The moisture-absorbing guard of claim 1, wherein the network of interconnected pores are distributed uniformly across a surface of the outer absorbent layer.

6. The moisture-absorbing guard of claim 1, wherein the network of interconnected pores comprises at least a portion of pores having a diameter of between about 5 mm and about 100 mm.

7. The moisture-absorbing guard of claim 1, wherein the base adhesive layer comprises an adhesive, wherein the adhesive is acrylic, silicone-based, hydrocolloid, polyurethane-based, a hydrogel, collagen-based, or a combination thereof.

8. The moisture-absorbing guard of claim 1, further comprising a protective liner attached to the base adhesive layer.

9. The moisture-absorbing guard of claim 8, wherein the protective liner comprises silicone-coated paper, a polyethylene film, a polypropylene film, wax-coated paper, a polyvinyl chloride film, fluoropolymer-coated paper, or a combination thereof.

10. The moisture-absorbing guard of claim 1, wherein the moisture-absorbing guard has a teardrop shape.

11. The moisture-absorbing guard of claim 1, wherein the moisture-absorbing guard has a butterfly shape.

12. The moisture-absorbing guard of claim 1, wherein the moisture-absorbing guard has an hourglass shape.

13. The moisture-absorbing guard of claim 1, wherein the moisture-absorbing guard has a rectangular shape.

14. The moisture-absorbing guard of claim 1, wherein the moisture-absorbing guard has an oval shape.

15. The moisture-absorbing guard of claim 1, wherein the moisture-absorbing guard consist of hypoallergenic materials.

16. The moisture-absorbing guard of claim 1, wherein the moisture-absorbing guard consist of biodegradable materials.

17. The moisture-absorbing guard of claim 1, wherein the moisture-absorbing guard is breathable.

18. The moisture-absorbing guard of claim 1, further comprising a scent-neutralizing layer.

19. The moisture-absorbing guard of claim 1, wherein the moisture-absorbing guard is free of active ingredients.

20. A method of using a moisture-absorbing guard comprising:
removing a protective liner of the moisture-absorbing guard,
positioning the moisture-absorbing guard onto a body of a user, and
applying the moisture-absorbing guard to the body of the user via a base adhesive layer of the moisture-absorbing guard,
wherein the moisture-absorbing guard further comprises an outer absorbent layer and a moisture-locking interior layer between the outer absorbent layer and the base adhesive layer.
